(19) United States
(12) Patent Application Publication
(10) Pub. No.: US 2006/0241568 A1
(43) Pub. Date: Oct. 26, 2006

(54) Arthroscopic chondrocyte implantation method and device

(76) Inventor: Gregory James Roger, New South Wales (AU)

Correspondence Address: JACOBSON HOLMAN PLLC, 400 SEVENTH STREET N.W., SUITE 600, WASHINGTON, DC, 20004, US

(21) Appl. No.: 10/545,225

(22) PCT Filed: Feb. 5, 2004
(86) PCT No.: PCT/AU04/00133

§ 371 Date: Jun. 26, 2006

(30) Foreign Application Priority Data
Feb. 12, 2003 (AU) 2003900620

(51) Int. Cl. A61K 9/22

(52) U.S. Cl. 604/891.1; 623/23.5

(57) ABSTRACT

A chondrocyte delivery device that has a main body (14) containing chondrocytes. The delivery device is at least partially insertable into a bone (18) of a patient and allowed or caused to elute the chondrocyte from the main body of the delivery device to damaged cartilage (17) in the region.

### FIELD OF THE INVENTION

The present invention relates to a surgical device and method and in particular to a device for use in the implantation of chondrocytes into cartilaginous tissue.

### BACKGROUND OF THE INVENTION

The ability of a knee or other joint to function normally depends on the presence of a smooth gliding surface. This is normally provided by articular cartilage which is a thin layer of tough tissue which covers the ends of bones where they meet in the joint. People with damage to or degeneration of this cartilage generally display symptoms which include joint locking, catching, localised pain, and swelling. In addition to pain and restricted mobility, chronic injuries to this cartilage over time may lead to debilitating osteoarthritis which can severely impact a person's normal activities.

Damaged cartilage has only a very limited capacity to heal itself. Methods of treatment do, however, depend on the severity of the degradation and the degree of activity that the patient wishes to pursue after treatment. Those patients that do choose to have no form of treatment can often expect their condition to worsen, with accelerated degeneration of the joint and onset of osteoarthrosis. For patients with less advanced conditions, non-surgical treatments, such as physical therapy can often provide some degree of relief. For patients with severe conditions, the only option generally available is surgery.

Current surgical techniques include arthroscopy in which an arthroscope is inserted into the joint and used to allow the surgeon to visualise the joint as he or she removes loose debris and fibres within the joint and/or trims away damaged cartilage. While this procedure is relatively minimally invasive it has been determined to be not effective in the long term.

Alternative techniques include disrupting the integrity of the underlying subchondral bone plate through techniques of drilling, abrasion, or microfracture. These techniques lead to local bleeding and transport of bone marrow cells into the bone which in turn leads to fibrous scar tissue generation. This fibrous tissue covers the area of chondral loss. While this treatment achieves improvement with little damage to the joint as a whole, the tissue is generally not as smooth as the cartilage it is replacing and the surface does not come up level with the surrounding cartilage. The fibrous tissue is also notably less durable than the cartilage tissue it is intended to replace. As such, these techniques are mainly palliative with effectiveness limited generally to a few years before recurrence of symptoms.

More recently, a technique of chondrocyte implantation has been developed as a method of treatment of joint degeneration. Chondrocytes are specialised cells that form the extracellular matrix that makes up cartilage. In this technique, chondrocytes can be taken from the patient, cultured and then re-implanted, or larger samples of chondrocyte tissue (cartilage) can be removed and then directly re-implanted into an area with deficient or damaged cartilage. The rationale of this latter procedure, commonly known as mosaicplasty is that the blocks of cartilage are removed from non-critical areas of weight bearing joints and used in diseased or damaged areas of cartilage.

An open surgical procedure is required to perform a chondrocyte implantation as the cultured chondrocytes are injected under a watertight patch that is sutured in place over the defect. The cells are held in place at the site of the defect by the patch. The patch is typically made of either cartilage tissue or an artificial matrix onto which chondrocyte cells have also been cultured. In either instance, an open surgical procedure is required so that the often large patch can be appropriately sutured to the surrounding tissue.

While autologous chondrocyte implantation has great potential and is likely to be very beneficial, opening a joint does have significant risks that have led to there being lower enthusiasm for the procedure than might have been expected. These risks include joint drying, increased infection potential, unanticipated joint damage, and significant patient pain.

The present invention is directed to a device and procedure for addressing at least some of these risks.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

### SUMMARY OF THE INVENTION

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

According to a first aspect, the present invention is a chondrocyte delivery device comprising a main body containing a plurality of chondrocytes, said delivery device at least partially insertable into a bone of a patient.

According to a second aspect, the present invention is a means for delivering a quantity of chondrocytes to a location within a patient, the delivery means being insertable into a bone of the patient, or a structure positioned therein, at or adjacent said location.

The delivery means typically comprises a main body having an internal chamber to store the chondrocytes.

According to a third aspect, the present application is directed to an apparatus for inserting a chondrocyte delivery means or device containing a quantity of chondrocytes into a bone of a patient, the apparatus comprising a syringe means having a needle adapted to contain the delivery means and insertable into the bone, and a plunger means adapted to expel the delivery means from the needle at a desired location in the bone.

In one embodiment, the main body of the delivery device comprises an internal chamber for storing the quantity of chondrocytes prior to and during implantation of the delivery device into the bone.

In one embodiment, the chamber of the delivery device and the delivery means can have at least one open or openable end through which the quantity of chondrocytes can exit the chamber. The main body of the delivery device or the delivery means may further include at least one aperture in a side wall through which the chondrocytes may migrate to surrounding bone and/or cartilaginous tissue. The openable end or the at least one aperture may initially be plugged by a sealing member. The sealing member may comprise a biodegradable substance such as a biodegradable polymer. Upon implantation of the chondrocyte delivery device or the delivery means in a bone of a patient, the polymeric material may degrade to allow release of the chondrocytes from the chamber.

In a further embodiment, the delivery means or device can have one or more additional chambers for storing bioactive substances.

In a preferred embodiment, the delivery means or device comprises a dart that is insertable into a bone or structure positioned therein. The dart can have a tapered leading end that facilitates its insertion into the bone or structure.

In a preferred embodiment, the delivery device or means is implanted into bone at a region that is in the vicinity of damaged or destroyed cartilaginous tissue, such that the chondrocytes may be released into said damaged or destroyed cartilaginous material.

Instead of cartilage at the location, a region of fibrous tissue can be formed at the site of the damaged cartilage. In a preferred embodiment, the fibrous tissue region is formed some time prior to the use of the chondrocyte delivery means. In a preferred embodiment, the fibrous tissue region is formed by a surgeon disrupting the subchondral bone plate at said location using a technique selected from the group comprising drilling, abrasion, or microfracture. In one embodiment, a technique of drilling known as the Steadman procedure can be used to ensure formation of the fibrous tissue. The disruption of the subchondral bone plate leads to local bleeding and transport of bone marrow cells into the bone which in turn leads to fibrous scar generation at said location. The fibrous tissue so formed typically covers the area of chondral loss.

In a further embodiment, the chondrocyte delivery means or device can be insertable through a structure comprising a fibrin mesh scaffold. The scaffold can be used to raise the level of the layer of fibrous tissue at said location to level or substantially level with the surrounding cartilage.

In a preferred embodiment, the delivery means or device is insertable into bone below the fibrous layer and/or the fibrin mesh scaffold, if present. The delivery means is preferably insertable such that the chondrocytes seed the area contained by the fibrous layer and/or fibrin scaffold with cells that form living cartilage. This living cartilage will over time preferably replace the fibrous tissue.

In a further embodiment, the delivery means or device can be insertable in an orientation that is substantially perpendicular to the surface of the bone. In this case, the chondrocyte cells can preferably elute from the chamber through an end of the delivery means or device that is positioned, following insertion, at or adjacent the surface of the fibrous tissue and/or bone.

In another embodiment, the delivery means or device can be insertable in a non-perpendicular or oblique orientation to the surface of the bone. In this case, the chondrocyte cells can elute from the chamber through a fissure running at least a portion of the length of a sidewall of the delivery means or device.

The delivery means or device are preferably insertable at the desired location in the patient without the necessity to perform an open operation, in which the joint is fully exposed, with its attendant risks of drying and infection.

Where substantially perpendicular insertion is employed, a guided placement technique could be employed to place the delivery means or device within the bone. In the case of oblique orientation, the placement of the delivery means or device can be visualised using an arthroscope.

In one embodiment, the delivery means or device further comprises an osmotic pump to assist in expelling the cells from the chamber following insertion. In one embodiment, the osmotic pump can be positioned at an end of the delivery means or device. In one embodiment, the osmotic pump can be positioned in the tapered leading end of the delivery means or device.

The delivery means or device is preferably formed from a biocompatible material. In one embodiment, the delivery means or device can be formed from a biocompatible metal or metal alloy. In one embodiment, the delivery means or device can be formed from titanium.

In a further embodiment, the main body of the delivery means or device can be formed from a bioresorbable material. The bioresorbable material can be selected from the group comprising calcium phosphate, hydroxyapatite, and PLLA. In this embodiment, bioactive compounds are bondable to the resorbable material and so released into said location on resorption of the main body. The main body can be adapted to resorb over a period of hours, days, weeks, months or longer.

The delivery means or device can have a trailing end distal the preferably tapered leading end. The delivery means or device can be insertable such that the trailing end is substantially coincident with the surface of the fibrous tissue. In another embodiment, the delivery means or device can be insertable such that the trailing end is not coincident with the surface of the fibrous tissue. In one embodiment, the trailing end can be positioned well within the surface of the fibrous tissue. Where the trailing end is positioned beneath the fibrous tissue, a cavity is preferably formed into which cells could elute from the chamber of the main body.

In another embodiment, the main body could be inserted into the bone and then retracted a distance shorter than its length. Such an insertion technique would preferably result in formation of a cavity coincident with the leading end of the delivery means or device into which cells could elute from the chamber. This may be particularly advantageous where the main body is inserted obliquely into the bone.

In one embodiment, the trailing end of the delivery means or device can have a fibrin cap that serves to preferably seal the surface of the fibrous layer behind the inserted delivery means or device.

In one embodiment of the apparatus of the third aspect, the needle can be formed of stainless steel and suitable for insertion into the fibrous tissue and bone of the patient. The needle can have a leading tapered end to facilitate its insertion into a bone of the patient. The diameter of the needle can expand moving back along its length away from the leading tapered end. During use, such a needle would form a longitudinal orifice in the bone, with the diameter of the orifice decreasing along its length inwardly away from the surface of the bone.

As the needle typically has a larger diameter than the diameter of the main body of the delivery means or device contained within it, on removal of the needle, a cavity will be present around the main body into which the cells may elute.

In one embodiment, the syringe means can be used in conjunction with an imaging technique, such as X-ray radiography, that would provide imaging of the needle as it is passed into the bone. As such, the syringe means could be utilised without the necessity to perform an open operation on the patient.

In a preferred embodiment, the delivery means or device is insertable in the knee joint of a patient. It will, however, be envisaged that the delivery means or device is insertable in another joint of the patient.

In a preferred embodiment, the delivery means or device is insertable in a knee joint so as to deliver chondrocyte cells to a location where articular cartilage is damaged or degraded.

In a further aspect, the present application is directed to a method of delivering chondrocyte cells to a location within a patient, the method comprising:

(i) inserting a delivery means containing a quantity of chondrocyte cells to said location; and

(ii) allowing or causing the chondrocyte cells to elute from the delivery means.

In another aspect, the present application is directed to a method of delivering chondrocyte cells to a location within a patient, the method comprising:

(i) inserting a chondrocyte delivery device comprising a main body containing a plurality of chondrocytes to said location; and

(ii) allowing or causing the chondrocyte cells to elute from the main body of the delivery device.

The delivery means or device described in the above methods can have any one or more of the features defined herein.

In a preferred embodiment, the patient firstly undergoes a step of harvesting chondral tissue. This harvesting step is preferably performed arthoscopically. Following harvest, chondrocyte cells are cultured ready for implantation back into the patient at a later date.

During the arthroscopic procedure to harvest the chondral tissue, a step of disrupting the subchondral bone plate at said location using a technique selected from the group comprising drilling, abrasion, or microfracture, is preferably performed. In one embodiment, a technique of drilling known as the Steadman procedure can be used to ensure formation of fibrous tissue at the site of the damaged cartilaginous tissue. The disruption of the subchondral bone plate leads to local bleeding and transport of bone marrow cells into the bone which in turn leads to fibrous scar generation at said location. The fibrous tissue so formed preferably covers the area of chondral loss.

In a further embodiment, the method can include a step of inserting a fibrin mesh scaffold into the joint at the site of the damage. The scaffold can be used to raise the level of the resultant layer of fibrous tissue at said location to level or substantially level with the surrounding cartilage. The mesh can be held in position with resorbable darts driven into the bone of the patient around said location.

Following a time sufficient to allow formation of the fibrous tissue and/or culturing of the chondrocyte cells, a further arthroscopy can be performed. During this arthroscopy, the fibrous layer can be assessed and any further required surgery carried out. The method can also include a step prior to this of imaging the joint, such as by computer tomography (CT), to confirm readiness for the joint to undergo the further arthroscopy. During or subsequent to this further arthroscopy, the step of inserting the delivery means or device into the joint can be performed.

In a further aspect, the present application is directed to a method of forming fibrous tissue at a site of damaged cartilaginous tissue of a bone joint, the method comprising the steps of:

(i) violating the subchondral bone plate at said site using a technique selected from the group comprising drilling, abrasion, or microfracture; said technique leading to local bleeding and transport of bone marrow cells into the bone and to subsequent formation of fibrous scar tissue; and

(ii) positioning a biocompatible mesh scaffold at said site;

wherein said scaffold raises the level of the resultant fibrous tissue to level or substantially level with the surrounding cartilage.

In this aspect, step (i) can comprise a technique of drilling known as the Steadman technique.

In this aspect, step (ii) is preferably performed after said technique but prior to formation of the fibrous tissue.

The biocompatible mesh scaffold can comprise a fibrin mesh scaffold. The mesh can be held in place once at said site by one or more resorbable darts driven into the bone of the patient around said site.

According to a further aspect, the present application is a mesh scaffold for use in a process of fibrous scar formation at a site of damaged cartilaginous tissue of a bone joint.

In this aspect, the mesh scaffold is preferably a fibrin mesh scaffold.

The present application describes a device and method of relatively minimally invasively delivering a suitable quantity of chondrocyte cells to a desired location in a bone of a patient, such as a knee joint. Still further, the present application describes a device and method of forming a layer of fibrous tissue at a site of damaged or destroyed cartilaginous tissue in a joint.

### BRIEF DESCRIPTION OF THE DRAWINGS

By way of example only, a preferred embodiment of the invention is now described with reference to the accompanying drawings, in which:

FIG. 1 is a simplified view of a bone joint surface having a layer of fibrous tissue extending across a site of damage in articular cartilage;

FIG. 2 is a simplified view of one embodiment of a delivery means according to the present invention inserted into the bone surface depicted in FIG. 1;

FIG. 3 is a simplified view of another embodiment of an apparatus for inserting a delivery means into the bone surface of a patient;

FIG. 4 is a simplified view of the embodiment of the delivery means depicted in FIG. 3 following its placement in the bone surface; and

FIG. 5 is a simplified enlarged view of another embodiment of a delivery means according to the present invention.

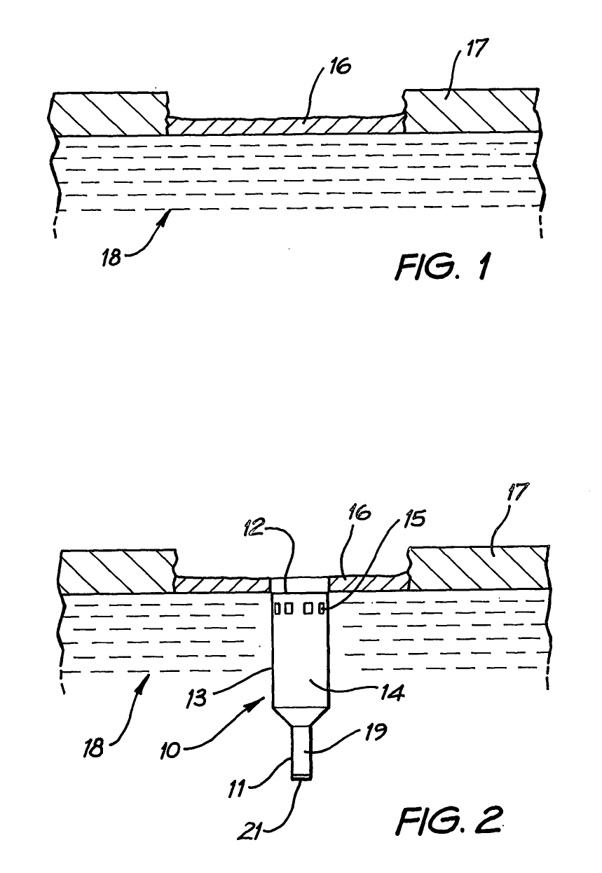

### PREFERRED MODE OF CARRYING OUT THE INVENTION

One embodiment of a delivery device in the form of a dart for relatively minimally invasively delivering a suitable quantity of chondrocyte cells to a desired location in a bone of a patient, such as a knee joint, is depicted generally as 10 in FIG. 2. In any typical operation, it will be appreciated that one or more dart 10 could be inserted into the joint.

In FIG. 2, the dart 10 has a leading end 11, a trailing end 12 and a titanium container body 13 extending between the respective ends. Disposed within the body 13 is a chamber 14 that can house a quantity of cultured chondrocyte cells.

In the depicted embodiment, the chamber 14 has a number of openings 15 through which the quantity of chondrocytes can elute from the chamber 14 following insertion of the dart 10.

Also, as depicted in FIG. 2, the dart 10 has an osmotic pump 19 housed within its leading end that is used to expel the cells from the chamber 14 following insertion of the dart 10. A semi-permeable membrane 21 is provided at the leading end of the dart 10 to allow operation of the osmotic pump 19.

While not depicted, it will be envisaged that the dart 10 could house other chambers for other bioactive substances that could also be released into the joint following its insertion.

In the embodiment depicted in FIG. 5, the delivery device is in the form of a bioresorbable cylindrical shell 30. The shell 30 houses a chamber 31 that can contain a quantity of chondrocyte cells. One end of the shell 30 can have a plug 32 that is also bioresorbable but at a rate that is typically quicker than that of the shell 30. Following insertion, the plug 32 can dissolve, preferably within about 24 hours, thereby allowing the chondrocyte cells to elute into the joint. Again, it will be appreciated that in any typical operation more than one shell 30 could be inserted into the joint.

The chamber 31 of the shell 30 can be sealed at its end distal the plug 32 with a fibrin cap 33.

In the embodiment depicted in FIG. 5, the bioresorbable material of the shell 30 can be selected from the group comprising calcium phosphate, hydroxyapatite, and PLLA. In this embodiment, bioactive compounds can be bonded to the resorbable material and so released into said location on resorption of the shell 30.

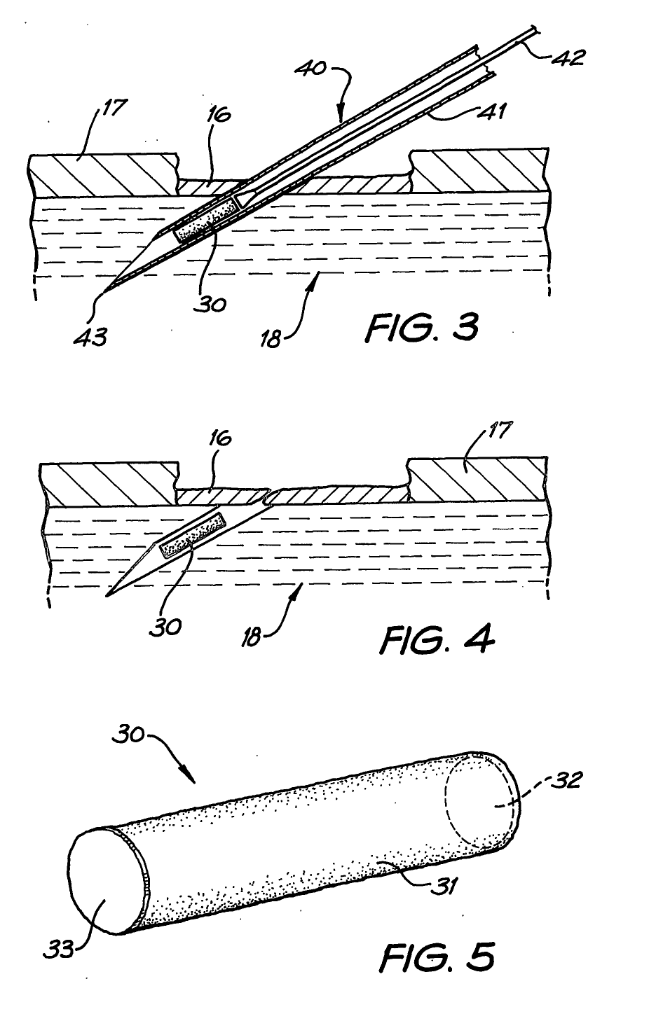

FIG. 3 depicts one embodiment of an apparatus 40 useable for inserting one or more shells 30 into a bone of a patient, such as a knee joint. While depicted being used in conjunction with shell 30, it will be appreciated that the apparatus 10 could be used in conjunction with dart 10 if desired. As depicted, the apparatus 40 comprises a syringe having a stainless steel needle 41 adapted to contain the shell 30 that is insertable into the bone, and a plunger 42 that is adapted to expel the dart 10 from the needle 41 at a desired location in the bone 18.

The needle 41 has a leading tapered end 43 to facilitate its insertion into the bone 18 of the patient. The diameter of the needle 41 also expands moving back along its length away from the leading tapered end 43. During use, the needle 41 forms a longitudinal orifice in the bone, with the diameter of the orifice decreasing along its length inwardly away from the surface of the bone 18.

As the needle 41 has a larger diameter than the shell 30 contained within it, on removal of the needle, a cavity will be present around the shell 30 into which the cells will be able to elute.

The apparatus 40 can be used in conjunction with an imaging technique, such as X-ray radiography, that would provide imaging of the needle 41 as it was passed into the bone 18. As such, the apparatus 40 can be utilised without the necessity to perform an open operation on the patient.

As is depicted in the drawings, the dart 10 or shell 30 is adapted to be inserted beneath a fibrous layer of tissue 16 that has been grown at the site of a defect in the cartilage 17 and positioned in the underlying bone 18.

As described above, the present application is also directed to a further invention comprising a method of delivering chondrocyte cells to a location within a patient. The method comprises the steps of:

(i) inserting a delivery means, such as dart 10 or shell 30, containing a quantity of chondrocyte cells to location, such as a knee joint; and

(ii) allowing or causing the chondrocyte cells to elute from the dart 10 or shell 30.

Prior to the delivery of the dart 10 or shell 30 into the patient, the patient will have typically firstly undergone an operation so as to harvest chondral tissue. This harvesting step would typically be performed arthoscopically. Following harvest, chondrocyte cells are cultured ready for implantation back into the patient at a later date.

During the arthroscopic procedure to harvest the chondral tissue, a step of violating the subchondral bone plate at the location using a technique selected from the group comprising drilling, abrasion, or microfracture, is also performed. In one embodiment, a technique of drilling known as the Steadman procedure is used to ensure formation of a layer of fibrous tissue 16 at the site of the damaged cartilaginous tissue. The violation of the subchondral bone plate leads to local bleeding and transport of bone marrow cells into the bone 18 which in turn leads to fibrous scar generation at the location. The layer of fibrous tissue 16 so formed preferably covers the area of chondral loss.

While not depicted but as is defined above, a fibrin mesh scaffold can be inserted into the joint at the site of the damage. The scaffold can be used to raise the level of the resultant layer of fibrous tissue 16 at the location to level or substantially level with the surrounding cartilage 17. The mesh can be held in position with resorbable darts driven into the bone 18 of the patient around the site.

Following a time sufficient to allow formation of the fibrous tissue 16 and/or culturing of the chondrocyte cells, a further arthroscopy can be performed. During this arthroscopy, the fibrous layer 16 can be assessed and any further required surgery carried out. The method can also include a step prior to this of imaging the joint, such as by computer tomography (CT), to confirm readiness for the joint to undergo the further arthroscopy. During or subsequent to this further arthroscopy, the step of inserting the dart 10 or shell 30 can be performed.

Once inserted, the dart 10 or shell 30 serves to deliver the chondrocytes into the joint and so seed the area contained by the fibrous layer 16 and/or fibrin scaffold with cells that form living cartilage. This living cartilage will over time preferably replace the fibrous tissue 16 and repair the defect in the cartilage 17.

As depicted in FIG. 2, the dart 10 can be insertable in an orientation that is substantially perpendicular to the surface of the bone 18.

In another embodiment, the dart 10 can be insertable in a non-perpendicular or oblique orientation to the surface of the bone 18. In this case, the chondrocyte cells can elute from the chamber 14 through a fissure (not depicted) running at least a portion of the length of a sidewall of the dart 10.

Where substantially perpendicular insertion is employed, a guided placement technique can be employed to place the dart 10 within the bone 18. In the case of oblique orientation, the placement of the dart 10 can be visualised using an arthroscope.

Once inserted, the trailing end 12, being formed of a fibrin cap, can be substantially coincident with the surface of the fibrous tissue 16 as is depicted in FIG. 2. It will, however, be appreciated that the dart 10 can be insertable such that the trailing end 12 is not coincident with the surface of the fibrous tissue 16. For example, the trailing end 12 can be positioned well beneath the surface of the fibrous tissue 16. When the trailing end 12 is positioned beneath the fibrous tissue, a cavity is preferably formed into which cells could elute from the chamber 14.

As depicted in FIGS. 3 and 4, the shell 30 can be inserted obliquely into the bone 18 using the syringe apparatus 40. In this arrangement, the needle 41 could be inserted into the bone 18 and then retracted a relatively short distance, prior to the step of expelling the shell 30 from the needle 41. Such an insertion technique results in formation of a cavity coincident with the leading end of the shell 30 into which cells could elute from the chamber.

The dart 10 or shell 30 is preferably insertable at the desired location in the patient without the necessity to perform an open operation, in which the joint is fully exposed, with its attendant risks of drying and infection.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A chondrocyte delivery device comprising a main body containing a plurality of chondrocytes, said delivery device at least partially insertable into a bone of a patient.

2. The chondrocyte delivery device of claim 1 wherein the main body comprises an internal chamber to house the chondrocytes.

3. The chondrocyte delivery device of claim 2 wherein the main body has at least one open or openable end in fluid communication with the chamber.

4. The chondrocyte delivery device of claim 2 wherein the main body includes at least one aperture in a side wall, said at least one aperture in fluid communication with the chamber.

5. The chondrocyte delivery device of claim 4 wherein the at least one aperture is substantially sealed by a removable or biodegradable sealing member.

6. The chondrocyte delivery device of claim 2 wherein the main body has one or more additional chambers for storing bioactive substances.

7. The chondrocyte delivery device of any one of the preceding claims wherein the main body has a leading end and a trailing end wherein said leading end is tapered.

8. The chondrocyte delivery device of any one of the preceding claims, said delivery device insertable in a region of bone substantially adjacent a region of damaged or destroyed cartilaginous tissue.

9. The chondrocyte delivery device of any one of the preceding claims wherein the entire main body is implantable within a region of bone of a patient.

10. The chondrocyte delivery device of any one of the preceding claims wherein said device is insertable through a region of fibrous tissue adjacent said bone of the patient.

11. The chondrocyte delivery device of any one of claim 1 to 9 wherein the device is insertable through a structure comprising a fibrin mesh scaffold and said bone of the patient.

12. The chondrocyte delivery device of any one of the preceding claims wherein the main body further comprises an osmotic pump to pump the chondrocyte cells from the chamber following insertion into said bone.

13. The chondrocyte delivery device of any one of the preceding claims wherein the main body is formed from a biocompatible material including a biocompatible metal or metal alloy, including titanium.

14. The chondrocyte delivery device of any one of claims 1 to 12 wherein the main body is formed from a bioresorbable material including a material selected from the group comprising calcium phosphate, hydroxyapatite, and PLLA.

15. The chondrocyte delivery device of claim 7 wherein the trailing end of the delivery means or device has a fibrin cap.

16. The chondrocyte delivery device of any one of the preceding claims said device insertable in a region of bone of a knee joint of the patient.

17. A means for delivering a quantity of chondrocytes to a location within a patient, the delivery means being insertable into a bone of the patient, or a structure positioned therein, at or adjacent said location.

18. An apparatus for inserting a chondrocyte delivery device containing a quantity of chondrocytes into a bone of a patient, the apparatus comprising a syringe means having a needle adapted to contain the delivery device and insertable into the bone, and a plunger means adapted to expel the delivery device from the needle at a desired location in the bone.

19. The apparatus of claim 18 wherein the needle is formed of stainless steel and is suitable for insertion into the fibrous tissue and bone of the patient.

20. The apparatus of claim 18 or claim 19 wherein the needle has a leading tapered end to facilitate its insertion into a bone of the patient and wherein the diameter of the needle expands moving back along its length away from the leading tapered end.

21. The apparatus of any one of claims 18 to 20 wherein when in use, said needle forms a cavity in the bone, said cavity housing the chondrocyte delivery device.

22. A method of delivering chondrocyte cells to a location within a patient, the method comprising:
(i) inserting a chondrocyte delivery device comprising a main body containing a plurality of chondrocytes to said location; and
(ii) allowing or causing the chondrocyte cells to elute from the main body of the delivery device.

23. The method of claim 22 wherein the location comprises a region of damaged cartilage in a joint of the patient.

24. The method of claim 23 wherein the device is implanted into the bone substantially beneath or surrounding the region of damaged or destroyed cartilage.

25. The method of any one of claims 22 to 24 wherein the delivery device is inserted in an orientation that is substantially perpendicular to the surface of the bone and wherein the chondrocyte cells elute from an end of the device following insertion.

26. The method of any one of claims 22 to 24 wherein the device is inserted in an oblique orientation relative to the surface of the bone and wherein the chondrocyte cells elute from a fissure in at least a portion of the length of a sidewall of the main body.

27. The method of any one of claims 22 to 26 wherein the device is inserted as part of a mininally invasive procedure.

28. The method of claim 23 including a first step of harvesting chondral tissue from the region of damaged cartilage.

29. The method of claim 28 wherein chondrocyte cells are cultured from said chondral tissue for implantation into the patient.

30. The method of claim 28 wherein during the harvesting of the chondral tissue, the subchondral bone plate at the site of harvest is disrupted by a technique including drilling, abrasion, or microfracture.

31. The method of claim 30 wherein said disruption of the subchondral bone plate leads to the formation of fibrous tissue.

32. The method of claim 23 including the further step of inserting a fibrin mesh scaffold into the joint at the site of the damage to the cartilage.

33. The method of claim 32 wherein the scaffold is held in position with resorbable darts driven into the bone of the patient around the damaged cartilage.

34. A method of delivering chondrocyte cells to a location within a patient, the method comprising:
(i) inserting a delivery means containing a quantity of chondrocyte cells to said location; and
(ii) allowing or causing the chondrocyte cells to elute from the delivery means.

35. A method of forming fibrous tissue at a site of damaged cartilaginous tissue of a bone joint, the method comprising the steps of:
(i) violating the subchondral bone plate at said site using a technique selected from the group comprising drilling, abrasion, or microfracture; said technique leading to local bleeding and transport of bone marrow cells into the bone and to subsequent formation of fibrous scar tissue; and
(ii) positioning a biocompatible mesh scaffold at said site;
wherein said scaffold raises the level of the resultant fibrous tissue to level or substantially level with the surrounding cartilage.

36. The method of claim 35 wherein step (i) comprises a technique of drilling.

37. The method of claim 35 wherein step (ii) is performed after said technique to violate the subchondral bone plate but prior to formation of the fibrous tissue.

38. A mesh scaffold for use in a process of fibrous scar formation at a site of damaged cartilaginous tissue of a bone joint.

39. The mesh scaffold of claim 38 said scaffold comprising a fibrin mesh scaffold.
